(19) United States
(12) Patent Application Publication
(10) Pub. No.: US 2026/0154945 A1
(43) Pub. Date: Jun. 4, 2026

(54) MEDICAL IMAGE PROCESSING METHOD AND SYSTEM

(71) Applicant: AItewan BioMedical Technology Inc., Taipei City (TW)

(72) Inventors: Yu-Te WU, Taipei City (TW); Fu-Ming WANG, Taipei City (TW)

(21) Appl. No.: 19/089,309

(22) Filed: Mar. 25, 2025

(30) Foreign Application Priority Data
Dec. 4, 2024 (TW) 113147089

(51) Int. Cl. G06V 10/764; A61B 5/00; G06T 7/00; G06V 10/25

(52) U.S. Cl. CPC G06V 10/764; A61B 5/4887; G06T 7/0012; G06V 10/25; G06T 2207/30016; G06T 2207/30048; G06T 2207/30064; G06T 2207/30096; G06T 2207/30101; G06V 2201/031; G06V 2201/032; G06V 2201/07

(57) ABSTRACT

A medical image processing method includes a target detection process, a focusing area mask mapping process, and a target classification process. The target detection process is to detect a target from a medical image and to sense at least one characteristic information of the target in the medical image. The focusing area mask mapping process is to generate a focusing area mask containing a focusing area corresponding to the medical image, to superimpose the focusing area mask on the medical image to generate a superimposed medical image, and to compare the property of the target in the superimposed medical image with the property of the focusing area so as to generate a classification result. The target classification process is to perform a property classification on the target based on classification enhancement information to generate another classification result, when the target is located outside the focusing area mask.

### CROSS REFERENCE TO RELATED APPLICATIONS

This Non-provisional application claims priority under 35 U.S.C. § 119(a) on Patent Application No(s). 113147089 filed in Taiwan, Republic of China on Dec. 4, 2024, the entire contents of which are hereby incorporated by reference.

### BACKGROUND

### Technology Field

This disclosure relates to a medical image processing method and system. In particular, this disclosure relates to a medical image processing method and system that can accurately and quickly classify a target in the medical image with utilizing a focusing area mask related to the medical image and classification enhancement information.

### Description of Related Art

With the development of artificial intelligence (AI) technology, the application of AI technology in the interpretation of medical images, which has become increasingly common, is referred to the AI in medical imaging. However, there are still many problems about the AI in medical imaging to be solved. Regarding the brain tumor detection and classification, since there are many types of tumors that form in the patients' brains, it is basically very difficult to detect and classify all or most of the characteristics and types of brain tumors accurately and quickly by using a single medical imaging AI system.

When processing targets in medical images, the targets to be detected and classified include brain tumors and other targets, such as calcium points in heart blood vessels or nodules in the lungs. Therefore, it is more difficult to detect and classify different targets by using a single medical imaging AI system.

In addition, when using AI in medical imaging for target detection and classification, in addition to the above problems, another most common technical problem is the misjudgment of some non-targets that should not be detected, or the omission of targets that should be detected. Although, in recent years, relevant technicians have proposed some solutions to these problems, such as the preprocessing of images, and these solutions have improved the problems to a certain extent, there are still many issues to be improved in detection and classification for the targets having numerous properties (e.g. various types of brain tumors).

With current technology of AI in medical imaging, it is difficult to accurately and quickly detect and classify all the characteristics and properties of targets with a single medical imaging AI system. Therefore, the conventional medical imaging AI system is only used as a general reference for medical units or clinical physicians, and cannot be fully utilized in clinical medicine. Furthermore, the conventional medical imaging AI system cannot provide powerful clinical application in clinical medicine, so it is even more impossible to use the conventional medical imaging AI system for providing early warning of cases that require priority treatment to the medical personnel.

Therefore, it is desired to provide a medical image processing method and system that can accurately detect the target and quickly classify the target. It is also desired to provide a medical image processing method and system that can be suitable for the detection and classification of different targets.

### SUMMARY

An objective of this disclosure is to provide a medical image processing method and system that can be applied to different target and can quickly and accurately classify the target.

To achieve the above, a medical image processing method of this disclosure includes a target detection process, a focusing area mask mapping process, and a target classification process. The target detection process is to detect a target from a medical image and to sense at least one characteristic information of the target in the medical image. The focusing area mask mapping process is to generate a focusing area mask at least containing a focusing area corresponding to the medical image, to superimpose the focusing area mask on the medical image to generate a superimposed medical image, and to compare a property of the target in the superimposed medical image with a property of the focusing area so as to generate a classification result. The target classification process is to perform a property classification on the target based on at least one classification enhancement information to generate another classification result, when the target is located outside the focusing area mask, or when the target is located within the focusing area, and the characteristic information of the target is different from a preset characteristic information of the focusing area.

In one embodiment, the medical image is a brain medical image, and the target is a brain tumor.

In one embodiment, the focusing area mask is a benign tumor area mask, and the focusing area is a benign tumor area.

In one embodiment, the benign tumor area includes at least one of a meningioma area, a schwannoma area, and a pituitary tumor area.

In one embodiment, the brain tumor is a malignant brain tumor, and the malignant brain tumor includes at least one of a brain metastasis, a glioblastoma, and an anaplastic astrocytoma.

In one embodiment, the target detection process includes a first detection sub-process and a second detection sub-process.

In one embodiment, the target classification process includes a first classification sub-process and a second classification sub-process.

In one embodiment, the characteristic information of the target includes at least one of position information, shape information, annotation information, and size information.

In one embodiment, the characteristic information of the target includes at least one of texture information, grayscale information, and HU value information.

In one embodiment, the medical image is a chest and lung medical image, and the target is a lung nodule.

In one embodiment, the focusing area mask is a lung area mask, and the focusing area is a lung area.

In one embodiment, the lung area includes at least one of a lung apex area and a lung base area.

In one embodiment, the medical image is a cardiac medical image, and the target is a coronary artery calcium (CAC) point.

In one embodiment, the focusing area mask is a coronary artery area mask, and the focusing area is a coronary artery area.

In one embodiment, the coronary artery area includes at least one of a right coronary artery area, a left main coronary artery area, a left anterior descending artery area, and a left circumflex descending artery area.

In one embodiment, the medical image processing method further includes a priority case push process for pushing the characteristic information of the target and the classification result or the property of the target to a specific person or a specific device.

In one embodiment, the classification enhancement information includes the characteristic information sensed by the target detection process, or imported classification enhancement information from outside.

To achieve the above, a medical image processing system of this disclosure includes a target detection module, a focusing area mask mapping module, and a target classification module. The target detection module detects a target from a medical image and senses at least one characteristic information of the target in the medical image. The focusing area mask mapping module is connected to the target detection module. The focusing area mask mapping module generates a focusing area mask at least containing a focusing area corresponding to the medical image, superimposes the focusing area mask on the medical image to generate a superimposed medical image, and compares a property of the target in the superimposed medical image with a property of the focusing area so as to generate a classification result. The target classification module is connected to the target detection module and the focusing area mask mapping module. The target classification module performs a property classification on the target based on at least one classification enhancement information to generate another classification result, when the target is located outside the focusing area mask, or when the target is located within the focusing area, and the characteristic information of the target is different from a preset characteristic information of the focusing area.

In one embodiment, the medical image is a brain medical image, and the target is a brain tumor.

In one embodiment, the focusing area mask is a benign tumor area mask, and the focusing area is a benign tumor area.

In one embodiment, the benign tumor area includes at least one of a meningioma area, a schwannoma area, and a pituitary tumor area.

In one embodiment, the brain tumor is a malignant brain tumor, and the malignant brain tumor includes at least one of a brain metastasis, a glioblastoma, and an anaplastic astrocytoma.

In one embodiment, the target detection module includes a first detection sub-module and a second detection sub-module.

In one embodiment, the target classification module includes a first classification sub-module and a second classification sub-module.

In one embodiment, the characteristic information of the target includes at least one of position information, shape information, annotation information, and size information.

In one embodiment, the characteristic information of the target includes at least one of texture information, grayscale information, and HU value information.

In one embodiment, the medical image is a chest and lung medical image, and the target is a lung nodule.

In one embodiment, the focusing area mask is a lung area mask, and the focusing area is a lung area.

In one embodiment, the lung area includes at least one of a lung apex area and a lung base area.

In one embodiment, the medical image is a cardiac medical image, and the target is a coronary artery calcium (CAC) point.

In one embodiment, the focusing area mask is a coronary artery area mask, and the focusing area is a coronary artery area.

In one embodiment, the coronary artery area includes at least one of a right coronary artery area, a left main coronary artery area, a left anterior descending artery area, and a left circumflex descending artery area.

In one embodiment, the medical image processing system further includes a priority case push module for pushing the characteristic information of the target and the classification result or the property of the target to a specific person or a specific device.

In one embodiment, the classification enhancement information includes the characteristic information sensed by the target detection module, or imported classification enhancement information from outside.

### BRIEF DESCRIPTION OF THE DRAWINGS

The disclosure will become more fully understood from the detailed description and accompanying drawings, which are given for illustration only, and thus are not limitative of the present disclosure, and wherein:

FIG. 1 is a block diagram showing the flow of a medical image processing method according to an embodiment of this disclosure;

FIG. 2 is a perspective diagram showing a brain image marked with a section plane a1;

FIG. 2A is a schematic diagram showing a brain medical image referring to a slice in the axial view of the brain image of FIG. 2 along the section plane a1;

FIG. 3 is a perspective diagram showing a brain image marked with a section plane b1;

FIG. 3A is a schematic diagram showing a brain medical image referring to a slice in the axial view of the brain image of FIG. 3 along the section plane b1;

FIG. 3B is a schematic diagram showing a focusing area mask Mk with respect to the brain medical image of FIG. 3A, wherein a focusing area of the focusing area mask Mk includes a schwannoma area B_Ar1 and a meningioma area B_Ar2;

FIG. 3C is a schematic diagram showing a superimposed medical image generated by superimposing the focusing area mask Mk of FIG. 3B on the brain medical image of FIG. 3A;

FIG. 4 is a perspective diagram showing a brain image marked with a section plane c1;

FIG. 4A is a schematic diagram showing a brain medical image referring to a slice in the axial view of the brain image of FIG. 4 along the section plane c1;

FIG. 4B is a schematic diagram showing a focusing area mask Mk with respect to the brain medical image of FIG. 4A, wherein a focusing area of the focusing area mask Mk includes a meningioma area B_Ar2 and a pituitary tumor area B_Ar3;

FIG. 4C is a schematic diagram showing a superimposed medical image generated by superimposing the focusing area mask Mk of FIG. 4B on the brain medical image of FIG. 4A;

FIG. 5 is a perspective diagram showing a lung image marked with a section plane a2;

FIG. 5A is a schematic diagram showing a lung apex medical image referring to a slice in the axial view of the lung image of FIG. 5 along the section plane a2;

FIG. 5B is a schematic diagram showing a focusing area mask Mk with respect to the lung apex medical image of FIG. 5A, wherein a focusing area of the focusing area mask Mk includes a left lung apex area L_Ar1 and a right lung apex area L_Ar2;

FIG. 5C is a schematic diagram showing a superimposed medical image generated by superimposing the focusing area mask Mk of FIG. 5B on the lung medical image of FIG. 5A;

FIG. 6 is a perspective diagram showing a lung image marked with a section plane b2;

FIG. 6A is a schematic diagram showing a lung base medical image referring to a slice in the axial view of the lung image of FIG. 6 along the section plane b2;

FIG. 6B is a schematic diagram showing a focusing area mask Mk with respect to the lung base medical image of FIG. 6A, wherein a focusing area of the focusing area mask Mk includes a left lung base area L_Ar3 and a right lung base area L_Ar4;

FIG. 6C is a schematic diagram showing a superimposed medical image generated by superimposing the focusing area mask Mk of FIG. 6B on the lung medical image of FIG. 6A;

FIG. 7 is a block diagram showing the flow of a medical image processing method according to another embodiment of this disclosure;

FIG. 8 is a perspective diagram showing a cardiac image marked with a section plane a3;

FIG. 8A is a schematic diagram showing a cardiac medical image referring to a slice in the axial view of the cardiac image of FIG. 8 along the section plane a3;

FIG. 8B is a schematic diagram showing a focusing area mask Mk with respect to the cardiac medical image of FIG. 8A, wherein a focusing area of the focusing area mask Mk includes a left main coronary artery area H_Ar1 and a left anterior descending artery area H_Ar2;

FIG. 8C is a schematic diagram showing a superimposed medical image generated by superimposing the focusing area mask Mk of FIG. 8B on the cardiac medical image of FIG. 8A;

FIG. 9 is a perspective diagram showing a cardiac image marked with a section plane b3;

FIG. 9A is a schematic diagram showing a cardiac medical image referring to a slice in the axial view of the cardiac image of FIG. 9 along the section plane b3;

FIG. 9B is a schematic diagram showing a focusing area mask Mk with respect to the cardiac medical image of FIG. 9A, wherein a focusing area of the focusing area mask Mk includes a left anterior descending artery area H_Ar2, a left circumflex descending artery area H_Ar3, a right coronary artery area H_Ar4;

FIG. 9C is a schematic diagram showing a superimposed medical image generated by superimposing the focusing area mask Mk of FIG. 9B on the cardiac medical image of FIG. 9A;

FIG. 10 is a block diagram showing the flow of a medical image processing method according to another embodiment of this disclosure;

FIG. 11 is a block diagram showing the flow of a medical image processing method according to another embodiment of this disclosure;

FIG. 12 is a block diagram showing a medical image processing system according to an embodiment of this disclosure;

FIG. 13 is a block diagram showing a medical image processing system according to another embodiment of this disclosure; and

FIG. 14 is a block diagram showing a medical image processing system according to another embodiment of this disclosure.

### DETAILED DESCRIPTION OF THE DISCLOSURE

The present disclosure will be apparent from the following detailed description, which proceeds with reference to the accompanying drawings, wherein the same references relate to the same elements.

In order to avoid repeated explanation and redundancy, before specifically describing the embodiments of the present disclosure, the meanings of some terms are specifically defined below.

In the present disclosure, the term “medical image” can be a 2D or 3D medical image, and the image format of the medical image can be an MRI image, a CT image, etc.

The term “target” refers to the lesions to be detected in the medical images, such as tumors, nodules, calcium points, etc.

The term “focusing area mask” refers to a mask that at least includes a focusing area, and the mask is generated corresponding to a medical image.

The types of “benign tumor” or “malignant tumor” are not limited to those listed in the following embodiments of this disclosure, and the types of tumors listed in the following embodiments are only for explanations.

Although the meningioma area is defined as a benign tumor area, clinical experiences show that different types of malignant tumors may exist in the meningioma area. Therefore, this disclosure also proposes a specific solution therefor.

The term “classification enhancement information” includes the characteristic information sensed by the target detection process or module, or imported classification enhancement information from the outside. The “characteristic information” refers to at least one of position information, shape information, annotation information, size information, texture information, grayscale information, and HU value information.

The term “property of target” refers to the type of the target, or the benign or malignant of the tumor.

The medical image processing method according to embodiments of this disclosure will be described hereinafter with reference to the drawings.

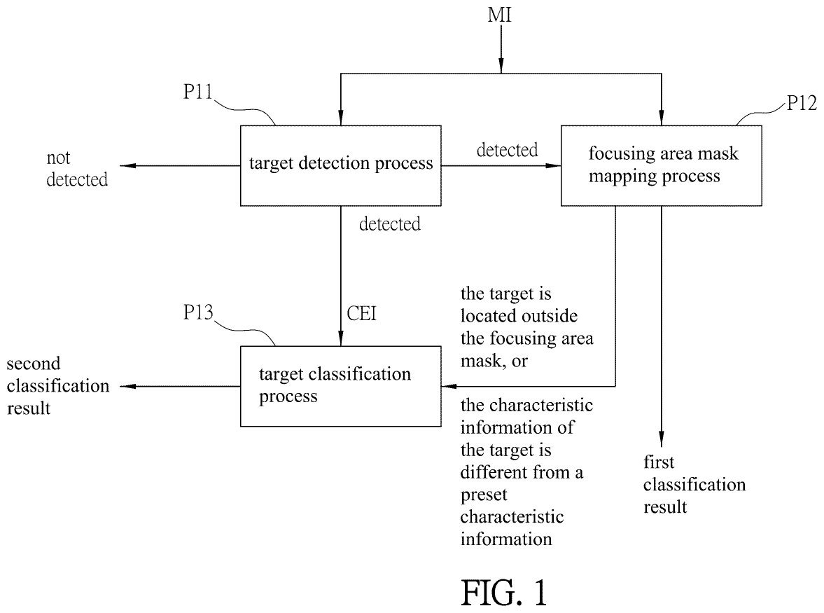

As shown in FIG. 1, the medical image processing method of this disclosure includes a target detection process P11, a focusing area mask mapping process P12, and a target classification process P13. In this embodiment, the medical image MI is individually inputted into the target detection process P11 and the focusing area mask mapping process P12.

Referring to FIG. 1, the target detection process P11 is to detect a target from a medical image MI and to sense at least one characteristic information of the target in the medical image MI. In this embodiment, the characteristic information can be, for example, at least one of position information, shape information, annotation information, size information, texture information, grayscale information, and HU value information. It should be noted that the characteristic information to be detected for specific targets may be different. In addition, at least one of the above-mentioned characteristic information can be provided as a classification enhancement information CEI for the target classification process P13.

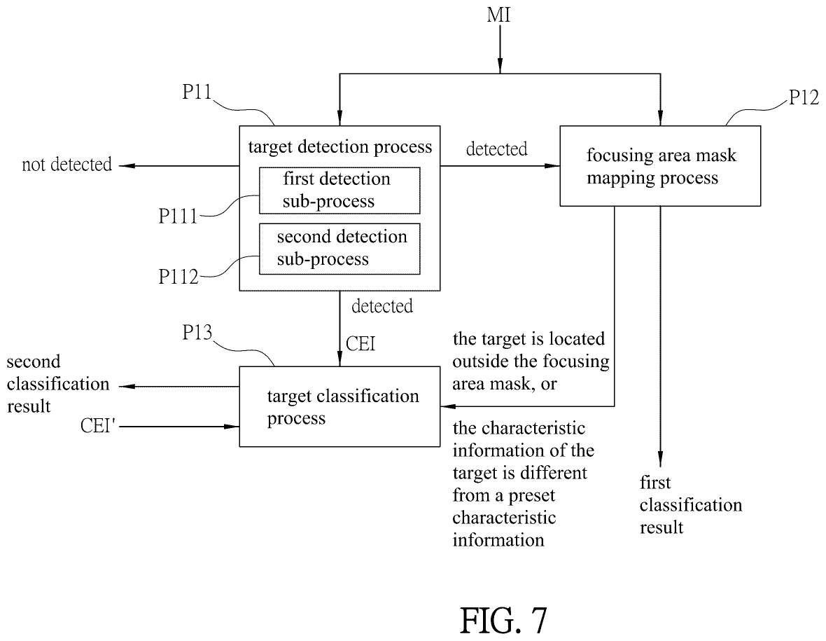

To be noted, different targets to be detected may correspond to different characteristic information to be sensed, and different characteristic information must be sensed by different detection procedures. As shown in FIG. 7, in the present disclosure, the target detection process P11 may include a first detection sub-process P111 and a second detection sub-process P112, and the different characteristic information, such as the position information and the annotation information, can be detected by the first detection sub-process P111 and the second detection sub-process P112 respectively. In practice, the first detection sub-process P111 or the second detection sub-process P112 can detect two or more types of characteristic information. For example, both of the annotation information and the size information can be detected by one of the first detection sub-process P111 and the second detection sub-process P112. In addition, the target detection process P11 may include any of other detection sub-processes (not shown).

When the target detection process P11 has found the target, the focusing area mask mapping process P12 can generate a focusing area mask Mk at least containing a focusing area corresponding to the medical image MI, superimpose the focusing area mask Mk on the medical image MI to generate a superimposed medical image MIs, and compare a property of the target in the superimposed medical image MIs with a property of the focusing area so as to generate a first classification result.

When the target detected by the target detection process P11 is located outside the focusing area mask Mk, or when the target by the target detection process P11 is located within the focusing area, and the characteristic information of the target is different from a preset characteristic information of the focusing area, the target classification process P13 is to perform a property classification on the target based on at least one classification enhancement information CEI, which is the characteristic information sensed by the target detection process P11, to generate a second classification result. To be noted, as shown in FIG. 7, in addition to using the characteristic information as the classification enhancement information CEI, it is possible to input additional classification enhancement information CEI′ (e.g. a patient's cancer information) from the outside. This cancer information can be used in the target classification process P13 to enhance the classification and thus more accurately classify whether the brain tumor is a metastatic tumor.

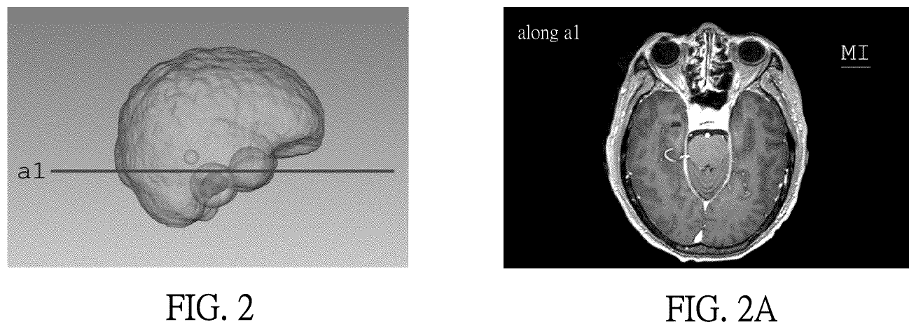

The medical image processing method will be further described hereinafter with reference to FIG. 2 to FIG. 4C, wherein, in this embodiment, the target is a brain tumor in a brain medical image.

It is well known that there are more than a dozen types of brain tumors in brain medical images, which are roughly divided into two categories: benign brain tumors and malignant brain tumors. Common benign brain tumors include meningioma, schwannoma, pituitary tumor, etc., and common malignant brain tumors include brain metastases, glioblastoma, anaplastic astrocytoma, etc. Accordingly, it is not easy to simply use an AI model to detect and classify all brain tumors. However, in order to more accurately and quickly detect and classify a brain tumor in a brain medical image, the present disclosure utilizes a benign tumor area mask as the focusing area mask Mk of this embodiment. That is, the focusing area is a benign tumor area, and the benign tumor area includes at least one of a meningioma area, a schwannoma area, and a pituitary tumor area.

FIG. 2 is a perspective diagram showing a brain image marked with a section plane a1, and FIG. 2A is a schematic diagram showing a brain medical image MI referring to a slice in the axial view of the brain image of FIG. 2 along the section plane a1. Referring to FIG. 1 and FIG. 2A, there is no brain tumor in the brain medical image MI in the slice along the section line a1, so the brain medical image MI of FIG. 2A is determined as not detected in the target detection process P11.

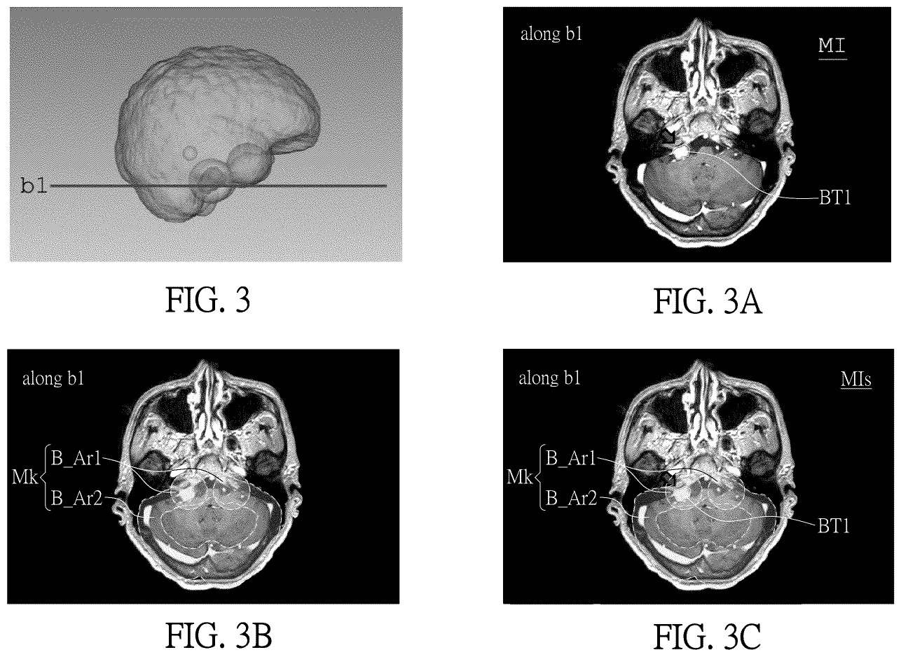

FIG. 3 is a perspective diagram showing a brain image marked with a section plane b1, and FIG. 3A is a schematic diagram showing a brain medical image referring to a slice in the axial view of the brain image of FIG. 3 along the section plane b1. Referring to FIG. 1 and FIG. 3A, when the brain medical image MI is detected by the target detection process P11, the brain tumor BT1 as the target can be detected. In this embodiment, the target detection process P11 can sense the position information or shape information of the target by means of object detection or image segmentation, and can sense the shape information or size information by marking. In addition, the U-Net model of Convolutional Neural Networks (CNNs) or the attention mechanism can be used to detect information such as contour information. It should be particularly noted that the sensing methods of various characteristic information are not limited to the aforementioned methods.

Although the target detection process P11 of this disclosure can sense the characteristic information related to the detected brain tumor, the aforementioned characteristic information alone is not enough to provide a complete description of the brain tumor BT1. In other words, if a complete description of the brain tumor BT1 is to be given, the name of the brain tumor BT1 and its benign or malignant property must be known. That is, the brain tumor BT1 must be classified quickly and accurately.

FIG. 3B is a schematic diagram showing a focusing area mask Mk with respect to the brain medical image of FIG. 3A, wherein a focusing area of the focusing area mask Mk includes a schwannoma area B_Ar1 and a meningioma area B_Ar2. Referring to FIG. 3B, the focusing area mask Mk is generated in the focusing area mask mapping process P12 corresponding to the medical image MI. The focusing area mask mapping process P12 is to superimpose the focusing area mask Mk on the medical image MI, thereby generating a superimposed medical image MIs (please refer to FIG. 3C). As shown in FIG. 3C, the brain tumor BT1 is located in the area indicated by B_Ar1. Since the area indicated by B_Ar1 is the schwannoma area, the property of the schwannoma area (the focusing area) can be used to compare with the property of the brain tumor in the superimposed medical image MIs, thereby generating a first classification result of the brain tumor BT1. In other words, the focusing area mask mapping process P12 can accurately and quickly determine that the brain tumor BT1 is a benign schwannoma (acoustic neuroma).

In this embodiment, although the benign brain tumor area can be used to compare and determine which type of benign brain tumor the target is, as for the meningioma area of the benign meningioma area, due to clinical experience, malignant brain tumors (e.g. metastatic tumors) may exist in the meningioma areas. Therefore, in the focusing area mask mapping process P12, when the characteristic information of the brain tumor in the meningioma area (e.g. the shape information of the brain tumor) is different from the preset characteristic information defined for the meningioma area, the image of the brain tumor in the meningioma area is transmitted to the target classification process P13, and the target classification process P13 performs a property classification on the target based on at least one characteristic information (classification enhancement information), thereby generating another classification result (i.e., a second classification result).

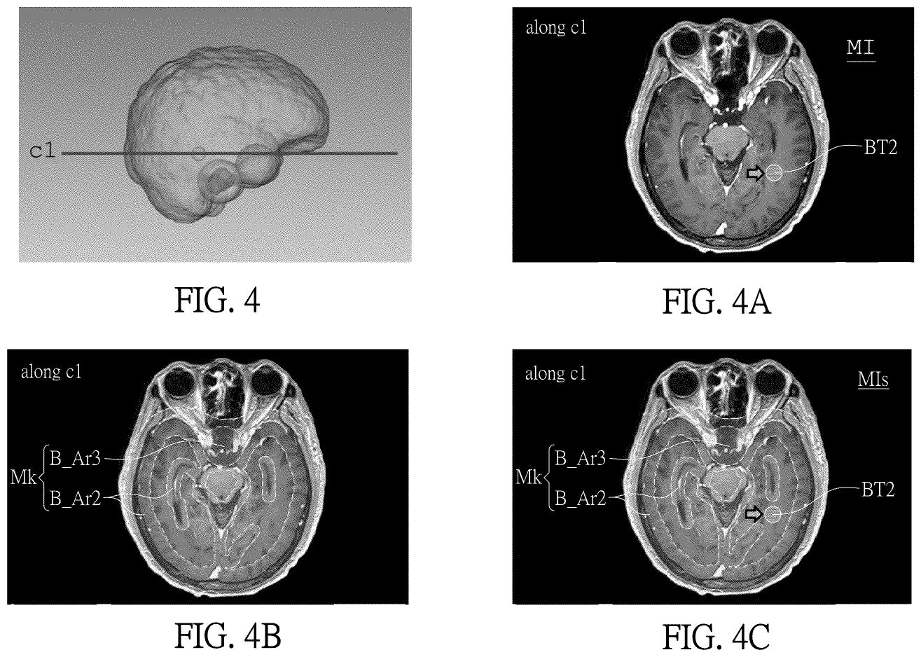

FIG. 4 is a perspective diagram showing a brain image marked with a section plane c1, and FIG. 4A is a schematic diagram showing a brain medical image MI referring to a slice in the axial view of the brain image of FIG. 4 along the section plane c1. Referring to FIG. 1 and FIG. 4A, the target detection process P11 can detect a target, which is a brain tumor BT2, from the brain medical image MI, and sense various characteristic information of the target.

As mentioned above, although the target detection process P11 of this disclosure can sense the characteristic information related to the detected brain tumor, the aforementioned characteristic information alone is not enough to provide a complete description of the brain tumor BT2. In other words, if a complete description of the brain tumor BT2 is to be given, the name of the brain tumor BT2 and its benign or malignant property must be known. That is, the brain tumor BT2 must be classified quickly and accurately.

FIG. 4B is a schematic diagram showing a focusing area mask Mk with respect to the brain medical image of FIG. 4A, wherein a focusing area of the focusing area mask Mk includes a meningioma area B_Ar2 and a pituitary tumor area B_Ar3. Referring to FIG. 4B, the focusing area mask Mk is generated in the focusing area mask mapping process P12 corresponding to the medical image MI. The focusing area mask mapping process P12 is to superimpose the focusing area mask Mk on the medical image MI, thereby generating a superimposed medical image MIs (please refer to FIG. 4C). As shown in FIG. 4C, the brain tumor BT2 is not located in the areas indicated by B_Ar2 and B_Ar3. In other words, the brain tumor BT2 is neither a meningioma nor a pituitary tumor.

As mentioned above, since brain tumor BT2 is neither a meningioma nor a pituitary tumor, it means that the location of brain tumor BT2 should not be within the focusing area of benign tumor. In other words, since the brain tumor BT2 is not located in the benign tumor area, it may be a malignant tumor. Therefore, it is necessary to enter the target classification process P13 for further classification. In the target classification process P13, the characteristic information sensed in the target detection process P11 is used as the classification enhancement information CEI. For malignant tumors, the preferred classification enhancement information CEI may include, for example, the shape information or the texture information. That is, in this embodiment, the target classification process P13 uses the classification enhancement information CEI, such as the shape information or texture information, to identify the brain tumor BT2. In this embodiment, the brain tumor BT2 is identified, by the target classification process P13, as a metastasis. That is, the second classification result indicates that the brain tumor BT2 is a malignant brain metastasis. To be noted, in addition to brain metastases, the possible identification of the malignant brain tumor may include glioblastomas or anaplastic astrocytoma, and the malignant brain tumors can be identified and classified by the target classification process P13 of the present disclosure.

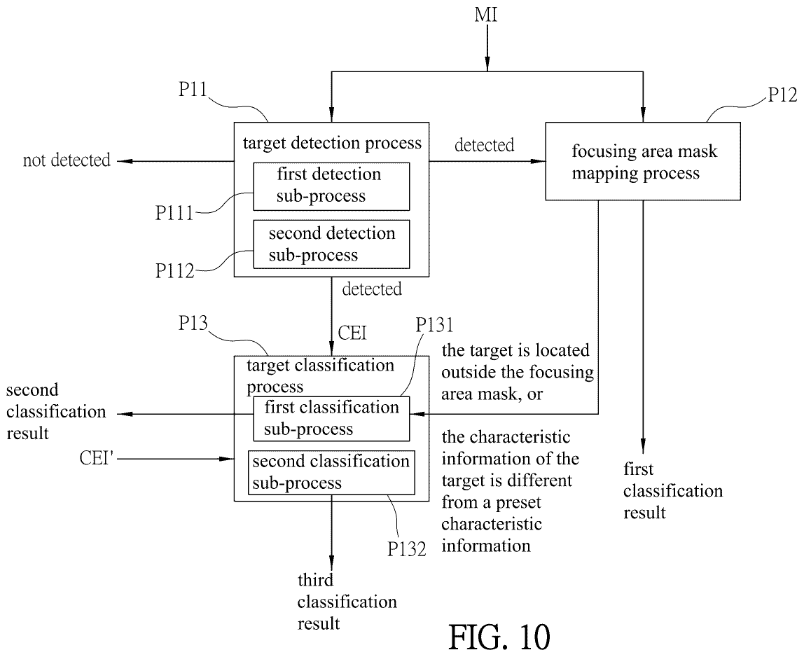

As shown in FIG. 10, the target detection process P11 of the present disclosure may include a first detection sub-process P111 and a second detection sub-process P112 for detecting and sensing multiple characteristic information of the target. When these characteristic information are used as the classification enhancement information of the target classification process P13, in addition to enabling the first classification sub-process P131 or the second classification sub-process P132 to perform accurate classification, these characteristic information can also be provided to a third classification sub-process (not shown) to determine whether the detected target is a pseudo target. For example, in the aforementioned brain medical image MI, if there are artifacts or spots caused by cerebral blood vessels, they may be detected simultaneously in the target detection process P11 of the present disclosure. Therefore, if the target classification process P13 further includes a third classification sub-process that specifically classifies artifacts and spots caused by cerebral blood vessels, the classification accuracy can be improved.

In the above embodiments, the medical image MI is a brain medical image, and the target is a brain tumor. From the above descriptions, it can be seen that the medical image processing method of the present disclosure can achieve rapid classification through a focusing area mask Mk associated with the medical image MI, or by using the classification enhancement information CEI to further accurately classify the target in the medical image MI. In brief, the medical image processing method of the present disclosure can achieve quick and accurate classification of targets.

In the following embodiments, the medical image MI is a lung medical image, and the target is a lung nodule. Since the basic functions of the target detection process P11, the focusing area mask mapping process P12, and the target classification process P13 of the following embodiments are roughly similar to those of the aforementioned embodiments in which the medical image MI is a brain medical image, only the differences therebetween will be described below.

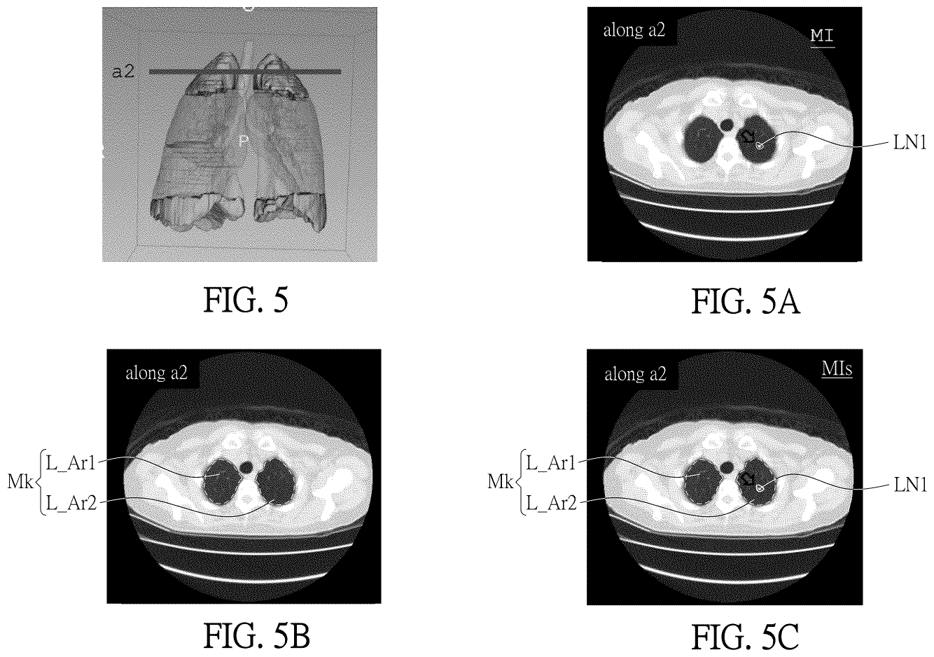

FIG. 5 is a perspective diagram showing a lung image marked with a section plane a2, and FIG. 5A is a schematic diagram showing a lung apex medical image MI referring to a slice in the axial view of the lung image of FIG. 5 along the section plane a2. Referring to FIG. 1 and FIG. 5A, the target detection process P11 can detect a target, which is a lung nodule LN1, from the lung apex medical image MI. In this embodiment, the characteristic information of the lung nodule LN1 sensed by the target detection process P11 may include, for example, texture information, grayscale information, size information, shape information, or the likes.

In order to more accurately and quickly detect and classify the lung nodule in the lung apex medical image MI, in this embodiment, a lung area mask is used as the focusing area mask Mk of this embodiment. The focusing area is the lung area, especially the lung apex area or the lung base area. Because in actual clinical judgment, the fibrotic lesions or calcified nodules in the apex area of the lung are often misdiagnosed as malignant. In addition, the lung base area of the lung is often misdiagnosed because it is located at the bottom of the chest cavity and adjacent to the diaphragm and abdominal organs.

FIG. 5B is a schematic diagram showing a focusing area mask Mk with respect to the lung apex medical image MI of FIG. 5A, wherein a focusing area of the focusing area mask Mk includes a left lung apex area L_Ar1 and a right lung apex area L_Ar2. Referring to FIG. 5B, the focusing area mask Mk is generated by the focusing area mask mapping process P12 corresponding to the lung apex medical image MI. The focusing area mask mapping process P12 can further superimpose the focusing area mask Mk on the lung apex medical image MI, thereby generating a superimposed medical image MIs (please refer to FIG. 5C). As shown in FIG. 5C, the lung nodule LN1 is located in the right lung apex area L_Ar2. However, in actual clinical practices, the lung apex area of the lung is often misjudged due to fibrotic lesions or calcified nodules. Therefore, in this embodiment, the target (lung nodule LN1) in the right lung apex area L_Ar2 must be classified again in the same manner as the meningioma area in the previous embodiment. That is, the target classification process P13 of the present disclosure must be performed based on the characteristic information. In other words, the characteristic information sensed by the target detection process P11 must be used as the classification enhancement information CEI in the target classification process P13 to perform a more accurate classification.

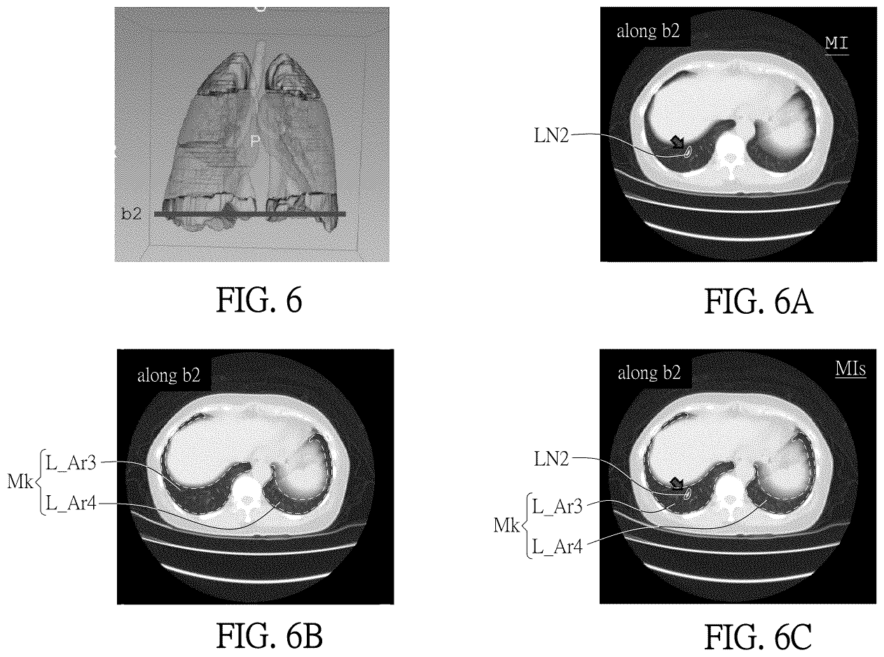

FIG. 6 is a perspective diagram showing a lung image marked with a section plane b2, and FIG. 6A is a schematic diagram showing a lung base medical image MI referring to a slice in the axial view of the lung image of FIG. 6 along the section plane b2. Referring to FIG. 1 and FIG. 6A, the target detection process P11 can detect a target, which is a lung nodule LN2, from the lung base medical image MI.

FIG. 6B is a schematic diagram showing a focusing area mask Mk with respect to the lung base medical image MI of FIG. 6A, wherein a focusing area of the focusing area mask Mk includes a left lung base area L_Ar3 and a right lung base area L_Ar4. Referring to FIG. 6B, the focusing area mask Mk is generated by the focusing area mask mapping process P12 corresponding to the lung base medical image MI. The focusing area mask mapping process P12 can further superimpose the focusing area mask Mk on the lung base medical image MI, thereby generating a superimposed medical image MIs (please refer to FIG. 6C). As shown in FIG. 6C, the lung nodule LN2 is located in the left lung base area L_Ar3. However, in actual clinical practices, the lung base area of the lung is often misjudged because it is located at the bottom of the chest cavity and adjacent to the diaphragm and abdominal organs. Therefore, in this embodiment, the target (lung nodule LN2) in the left lung base area L_Ar3 must be classified again in the same manner as the meningioma area in the previous embodiment. That is, the target classification process P13 of the present disclosure must be performed based on the characteristic information. In other words, the characteristic information sensed by the target detection process P11 must be used as the classification enhancement information CEI in the target classification process P13 to perform a more accurate classification.

In the above embodiments, the medical image MI is a lung medical image, and the target is a lung nodule. From the above descriptions, it can be seen that the medical image processing method of the present disclosure can achieve rapid classification through a focusing area mask Mk associated with the medical image MI, or by using the classification enhancement information CEI to further accurately classify the target in the medical image MI. In brief, the medical image processing method of the present disclosure can achieve quick and accurate classification of targets.

In the following embodiments, the medical image MI is a cardiac medical image, and the target is a coronary artery calcium (CAC) point. Since the basic functions of the target detection process P11, the focusing area mask mapping process P12, and the target classification process P13 of the following embodiments are roughly similar to those of the aforementioned embodiments in which the medical image MI is a brain medical image, only the differences therebetween will be described below.

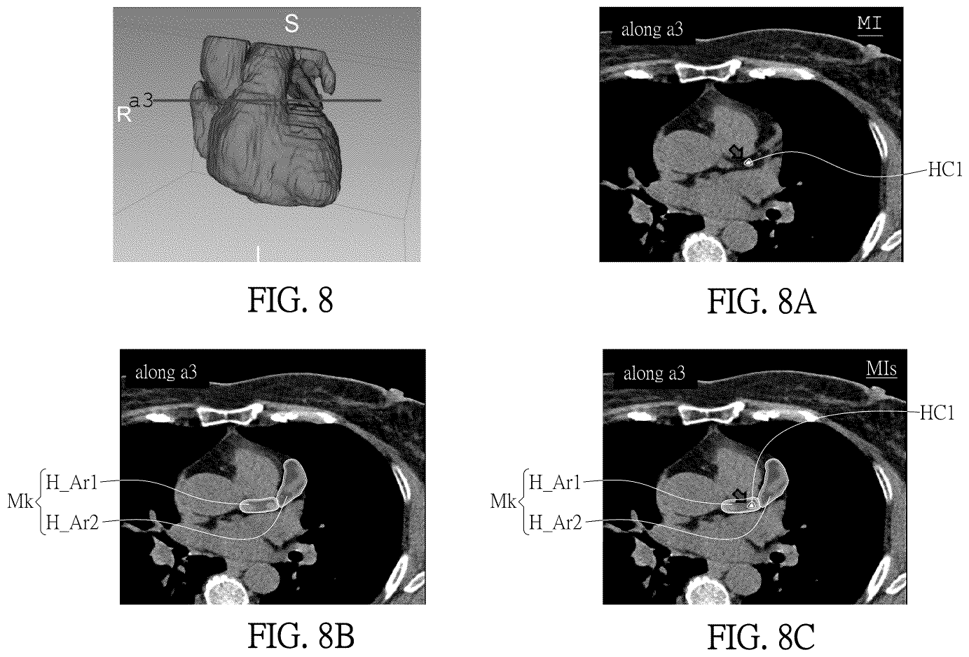

FIG. 8 is a perspective diagram showing a cardiac image marked with a section plane a3, and FIG. 8A is a schematic diagram showing a cardiac medical image referring to a slice in the axial view of the cardiac image of FIG. 8 along the section plane a3. Referring to FIG. 1 and FIG. 8A, the target detection process P11 can detect a target, which is a calcium point HC1, from the cardiac medical image MI. In this embodiment, the characteristic information of the calcium point HC1 sensed by the target detection process P11 may include, for example, at least one of texture information, grayscale information, size information, shape information, and HU value information.

In order to more accurately and quickly detect and classify the CAC point in the cardiac medical image MI, in this embodiment, a coronary artery area mask is used as the focusing area mask Mk of this embodiment. The focusing area is the coronary artery area, which may include a left main coronary artery area H_Ar1, a left anterior descending artery area H_Ar2, a left circumflex descending artery area H_Ar3, or a right coronary artery area H_Ar4.

FIG. 8B is a schematic diagram showing a focusing area mask Mk with respect to the cardiac medical image MI of FIG. 8A, wherein a focusing area of the focusing area mask Mk includes a left main coronary artery area H_Ar1 and a left anterior descending artery area H_Ar2. Referring to FIG. 8B, the focusing area mask Mk is generated by the focusing area mask mapping process P12 corresponding to the cardiac medical image MI. The focusing area mask mapping process P12 can further superimpose the focusing area mask Mk on the cardiac medical image MI, thereby generating a superimposed medical image MIs (please refer to FIG. 8C). As shown in FIG. 8C, the calcium point HC1 is located in the left main coronary artery area H_Ar1. In this case, if it is not necessary to further determine whether the calcium point HC1 a symptom of other diseases (e.g. lipid deposition), the following step of the target classification process P13 can be omitted.

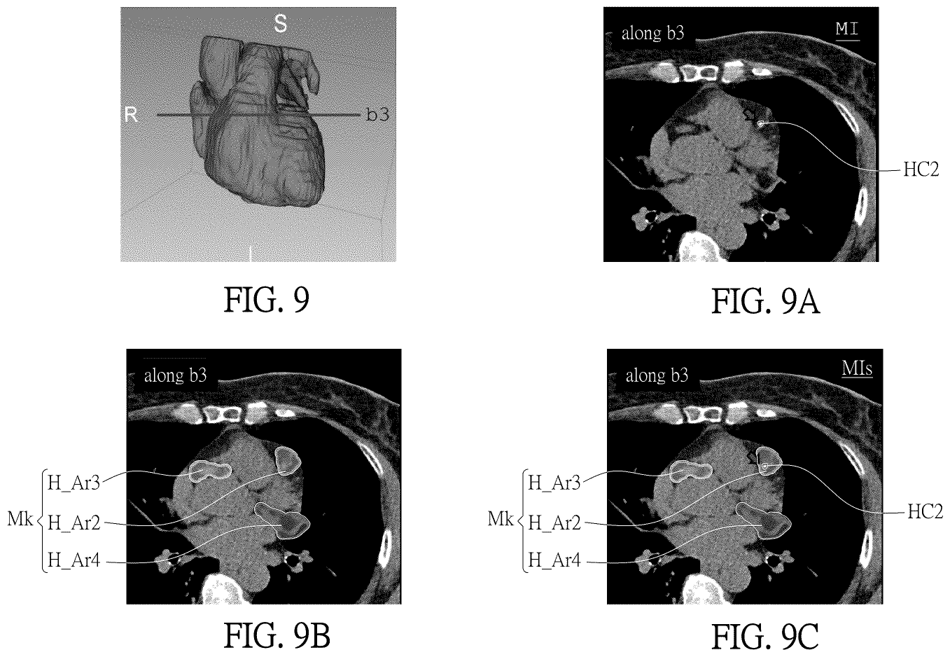

FIG. 9 is a perspective diagram showing a cardiac image marked with a section plane b3, and FIG. 9A is a schematic diagram showing a cardiac medical image MI referring to a slice in the axial view of the cardiac image MI of FIG. 9 along the section plane b3. Referring to FIG. 1 and FIG. 9A, the target detection process P11 can detect a target, which is a calcium point HC2, from the cardiac medical image MI.

FIG. 9B is a schematic diagram showing a focusing area mask Mk with respect to the cardiac medical image MI of FIG. 9A, wherein a focusing area of the focusing area mask Mk includes a left anterior descending artery area H_Ar2, a left circumflex descending artery area H_Ar3, and a right coronary artery area H_Ar4. Referring to FIG. 9B, the focusing area mask Mk is generated by the focusing area mask mapping process P12 corresponding to the cardiac medical image MI. The focusing area mask mapping process P12 can further superimpose the focusing area mask Mk on the cardiac medical image MI, thereby generating a superimposed medical image MIs (please refer to FIG. 9C).

As shown in FIG. 9C, the calcium point HC2 is located in the left anterior descending artery area H_Ar2. That is, the calcium point HC2 is a calcium point located in the left anterior descending artery area H_Ar2. In this case, if it is not necessary to further determine whether the calcium point HC2 is a symptom of other diseases (e.g. lipid deposition), the following step of the target classification process P13 can be omitted. However, in this embodiment, as shown in FIG. 10, the target classification process P13 may include a first classification sub-process P131 and a second classification sub-process P132. When the first classification sub-process P131 is not needed to further determine whether the calcification point HC2 is a symptom of other diseases, the second classification sub-process P132 may still be used to determine the calcification level classification (the third classification result) of the patient's coronary artery according to the calcification level of the calcium point HC1 and the calcium point HC2.

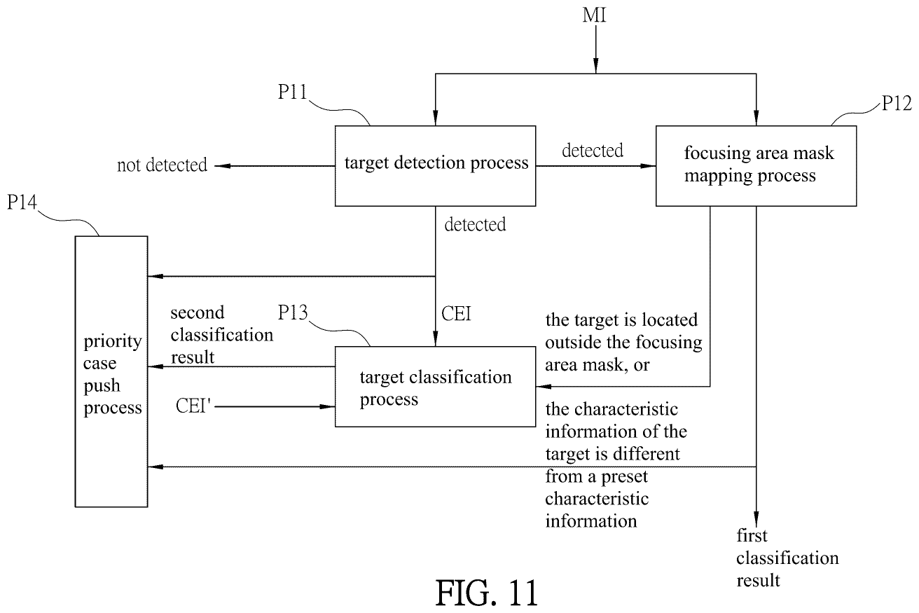

As mentioned above, the medical image processing method of the present disclosure can indeed classify a target accurately and quickly. That is, the medical image processing method of the present disclosure can sense various characteristic information of a target and the property of the target. In other words, the medical image processing method of the present disclosure can obtain the complete and accurate target description information. To be noted, only complete and accurate target description information can meet the actual clinical needs and can be used to quickly provide accurate information of warning cases or priority cases to a specific person (e.g. doctor) or a specific device (e.g. mobile phone). Therefore, as shown in FIG. 11, the medical image processing method of the present disclosure may further include a priority case push process P14.

The medical image processing system according to an embodiment of the present disclosure will be specifically described hereinafter. To be noted, since the medical image processing system of the present disclosure generally utilizes the aforementioned medical image processing method, in order to avoid redundancy, the following descriptions will only describe the differences between the system and the method, and other similar parts will be omitted.

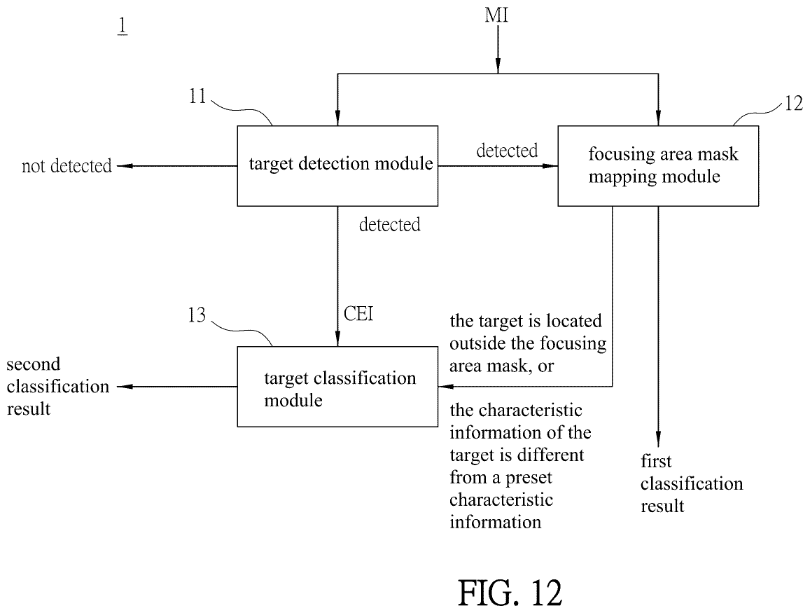

As shown in FIG. 12, the medical image processing system 1 of this disclosure includes a target detection module 11, a focusing area mask mapping module 12, and a target classification module 13.

The target detection module 11 is to detect a target from a medical image MI and to sense at least one characteristic information of the target in the medical image MI. The focusing area mask mapping module 12 is connected to the target detection module 11, and is to generate a focusing area mask Mk at least containing a focusing area corresponding to the medical image MI, to superimpose the focusing area mask Mk on the medical image MI to generate a superimposed medical image MIs, and to compare a property of the target in the superimposed medical image MIs with a property of the focusing area so as to generate a first classification result.

The target classification module 13 is connected to the target detection module 11 and the focusing area mask mapping module 12. When the target is located outside the focusing area mask Mk, or when the target is located within the focusing area, but the characteristic information of the target is different from a preset characteristic information of the focusing area, the target classification module 13 performs a property classification on the target based on at least one classification enhancement information CEI to generate another classification result (i.e., a second classification result).

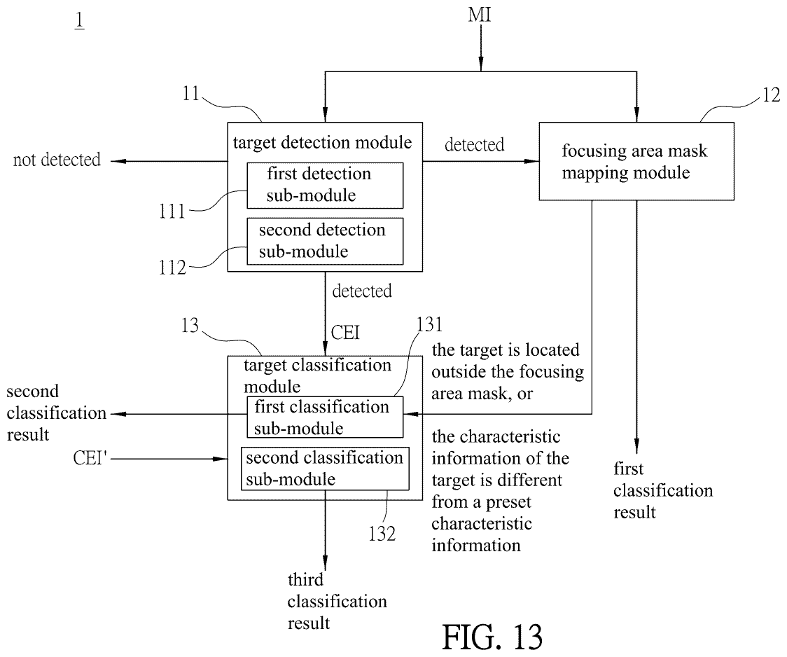

As shown in FIG. 13, the target detection module 11 may include a first detection sub-module 111 and a second detection sub-module 112, and the first detection sub-module 111 and the second detection sub-module 112 can be used to sense the characteristic information of different targets, respectively. In addition, as shown in FIG. 13, the target classification module 13 may include a first classification sub-module 131 and a second classification sub-module 132, and the first classification sub-module 131 and the second classification sub-module 132 can be used to perform the property classifications of different targets, respectively. To be noted, the target detection module 11 of the present disclosure, which includes the first detection sub-module 111 and the second detection sub-module 112, can sense multiple characteristic information of the target(s). When these characteristic information are used as classification enhancement information of the target classification module 13, in addition to enabling the first classification sub-module 131 or the second classification sub-module 132 to perform accurate classification, these characteristic information can further be provided to a third classification sub-module (not shown) to determine whether the detected target is a pseudo target. For example, in the aforementioned brain medical image MI, if there are artifacts or spots caused by cerebral blood vessels, they may be detected simultaneously by the target detection module 11 of the present disclosure. Therefore, if the target classification module 13 further includes a third classification sub-module that specifically classifies artifacts and spots caused by cerebral blood vessels, the classification accuracy can be improved.

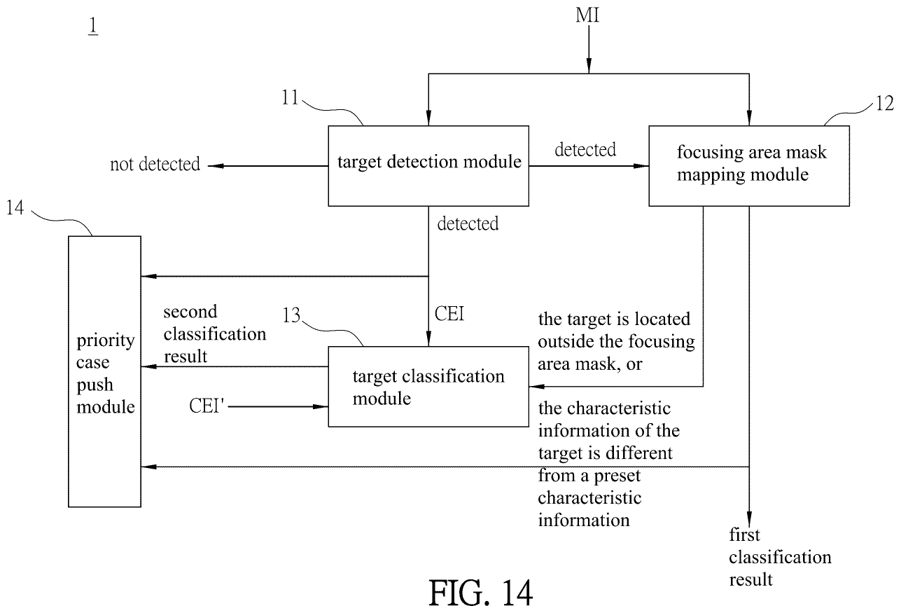

As shown in FIG. 14, the medical image processing system 1 of this disclosure may further include a priority case push module 14 for pushing the characteristic information of the target and the classification result or the property of the target to a specific person or a specific device.

Although the disclosure has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the disclosure.

What is claimed is:

1. A medical image processing method, comprising:
a target detection process for detecting a target from a medical image and sensing at least one characteristic information of the target in the medical image;
a focusing area mask mapping process for generating a focusing area mask at least containing a focusing area corresponding to the medical image, superimposing the focusing area mask on the medical image to generate a superimposed medical image, and comparing a property of the target in the superimposed medical image with a property of the focusing area so as to generate a classification result; and
a target classification process for performing a property classification on the target based on at least one classification enhancement information to generate another classification result, when the target is located outside the focusing area mask, or when the target is located within the focusing area, and the characteristic information of the target is different from a preset characteristic information of the focusing area.

2. The medical image processing method of claim 1, wherein the medical image is a brain medical image, and the target is a brain tumor.

3. The medical image processing method of claim 2, wherein the focusing area mask is a benign tumor area mask, and the focusing area is a benign tumor area.

4. The medical image processing method of claim 3, wherein the benign tumor area comprises at least one of a meningioma area, a schwannoma area, and a pituitary tumor area.

5. The medical image processing method of claim 2, wherein the brain tumor is a malignant brain tumor, and the malignant brain tumor comprises at least one of a brain metastasis, a glioblastoma, and an anaplastic astrocytoma.

6. The medical image processing method of claim 1, wherein the target detection process comprises a first detection sub-process and a second detection sub-process.

7. The medical image processing method of claim 1, wherein the target classification process comprises a first classification sub-process and a second classification sub-process.

8. The medical image processing method of claim 1, wherein the characteristic information of the target comprises at least one of position information, shape information, annotation information, and size information.

9. The medical image processing method of claim 1, wherein the characteristic information of the target comprises at least one of texture information, grayscale information, and HU value information.

10. The medical image processing method of claim 1, wherein the medical image is a chest and lung medical image, and the target is a lung nodule.

11. The medical image processing method of claim 10, wherein the focusing area mask is a lung area mask, and the focusing area is a lung area.

12. The medical image processing method of claim 11, wherein the lung area comprises at least one of a lung apex area and a lung base area.

13. The medical image processing method of claim 1, wherein the medical image is a cardiac medical image, and the target is a coronary artery calcium (CAC) point.

14. The medical image processing method of claim 13, wherein the focusing area mask is a coronary artery area mask, and the focusing area is a coronary artery area.

15. The medical image processing method of claim 14, wherein the coronary artery area comprises at least one of a right coronary artery area, a left main coronary artery area, a left anterior descending artery area, and a left circumflex descending artery area.

16. The medical image processing method of claim 1, further comprising:
a priority case push process for pushing the characteristic information of the target and the classification result or the property of the target to a specific person or a specific device.

17. The medical image processing method of claim 1, wherein the classification enhancement information comprises the characteristic information sensed by the target detection process, or imported classification enhancement information from outside.

18. A medical image processing system, comprising:
a target detection module detecting a target from a medical image and sensing at least one characteristic information of the target in the medical image;
a focusing area mask mapping module connected to the target detection module, wherein the focusing area mask mapping module generates a focusing area mask at least containing a focusing area corresponding to the medical image, superimposes the focusing area mask on the medical image to generate a superimposed medical image, and compares a property of the target in the superimposed medical image with a property of the focusing area so as to generate a classification result; and
a target classification module connected to the target detection module and the focusing area mask mapping module, wherein the target classification module performs a property classification on the target based on at least one classification enhancement information to generate another classification result, when the target is located outside the focusing area mask, or when the target is located within the focusing area, and the characteristic information of the target is different from a preset characteristic information of the focusing area.

19. The medical image processing system of claim 18, wherein the medical image is a brain medical image, and the target is a brain tumor.

20. The medical image processing system of claim 19, wherein the focusing area mask is a benign tumor area mask, and the focusing area is a benign tumor area.

21. The medical image processing system of claim 20, wherein the benign tumor area comprises at least one of a meningioma area, a schwannoma area, and a pituitary tumor area.

22. The medical image processing system of claim 19, wherein the brain tumor is a malignant brain tumor, and the malignant brain tumor comprises at least one of a brain metastasis, a glioblastoma, and an anaplastic astrocytoma.

23. The medical image processing system of claim 18, wherein the target detection module comprises a first detection sub-module and a second detection sub-module.

24. The medical image processing system of claim 18, wherein the target classification module comprises a first classification sub-module and a second classification sub-module.

25. The medical image processing system of claim 18, wherein the characteristic information of the target comprises at least one of position information, shape information, annotation information, and size information.

26. The medical image processing system of claim 18, wherein the characteristic information of the target comprises at least one of texture information, grayscale information, and HU value information.

27. The medical image processing system of claim 18, wherein the medical image is a chest and lung medical image, and the target is a lung nodule.

28. The medical image processing system of claim 27, wherein the focusing area mask is a lung area mask, and the focusing area is a lung area.

29. The medical image processing system of claim 28, wherein the lung area comprises at least one of a lung apex area and a lung base area.

30. The medical image processing system of claim 18, wherein the medical image is a cardiac medical image, and the target is a coronary artery calcium (CAC) point.

31. The medical image processing system of claim 30, wherein the focusing area mask is a coronary artery area mask, and the focusing area is a coronary artery area.

32. The medical image processing system of claim 31, wherein the coronary artery area comprises at least one of a right coronary artery area, a left main coronary artery area, a left anterior descending artery area, and a left circumflex descending artery area.

33. The medical image processing system of claim 18, further comprising:
a priority case push module pushing the characteristic information of the target and the classification result or the property of the target to a specific person or a specific device.

34. The medical image processing system of claim 18, wherein the classification enhancement information comprises the characteristic information sensed by the target detection module, or imported classification enhancement information from outside.
